(19) United States
(12) Patent Application Publication
(10) Pub. No.: US 2011/0301663 A1
(43) Pub. Date: Dec. 8, 2011

(54) TREATMENT OF A PELVIC CONDITION THROUGH INDIRECT ELECTRICAL STIMULATION

(75) Inventors: Guangjian Wang, Falcon Heights, MN (US); Edouard A. Koullick, Golden Valley, MN (US)

(73) Assignee: AMS RESEARCH CORPORATION, Minnetonka, MN (US)

(21) Appl. No.: 13/201,828

(22) PCT Filed: Feb. 16, 2010
(86) PCT No.: PCT/US10/24287

§ 371 Date: Aug. 16, 2011

Related U.S. Application Data
(60) Provisional application No. 61/153,362, filed on Feb. 18, 2009.

(51) Int. Cl. A61N 1/36

(52) U.S. Cl. 607/41; 607/46

(57) ABSTRACT

Embodiments of the invention are generally directed to a method of treating a pelvic condition of a patient through the indirect stimulation of a pelvic neuromuscular structure of the patient. In one embodiment of the method, an electrical stimulation signal is applied to a first neuromuscular structure of the patient. The electrical stimulation signal is communicated through a neural pathway to a second pelvic neuromuscular structure of the patient. The second neuromuscular structure is stimulated in response to the electrical stimulation signal communicated through the neural pathway. The pelvic condition is treated in response to the stimulation of the second pelvic neuromuscular structure.

### FIELD OF THE INVENTION

Embodiments of the present invention generally relate to the treatment of a pelvic condition of a patient through the indirect stimulation of a pelvic neuromuscular structure of the patient.

### BACKGROUND

Electrical stimulation systems have been used to deliver electrical stimulation therapy to patients to treat a variety of pelvic symptoms or pelvic conditions such as urinary incontinence. A typical electrical stimulation system includes one or more implantable medical leads coupled to an external or implantable electrical stimulator. The implantable medical lead may be percutaneously or surgically implanted in a patient on a temporary or permanent basis such that at least one stimulation electrode is positioned at a target stimulation site. The one or more electrodes may deliver electrical stimulation therapy to the target stimulation site in the form of electrical signals.

The target stimulation site of prior art pelvic condition treatment methods is generally directly related to the pelvic condition or symptom to be treated. For instance, prior art methods stimulate the sacral nerve at a location of S3 sacral foramen for the treatment of overactive bladder; the pudendal nerve at a location of Alcock's canal for the treatment of overactive bladder; a peri-urethral neuromuscular structure, such as Accessa® for the treatment of interstitial cystitis and overactive bladder; the internal urinary sphincter muscle for the treatment of urinary incontinence (see for example, U.S. Pat. Nos. 6,354,991, 6,652,449, 6,712,772 and 6,862,480); and the sacral nerve for the treatment of urinary retention.

### SUMMARY

Embodiments of the invention are generally directed to a method of treating of a pelvic condition of a patient through the indirect stimulation of a pelvic neuromuscular structure of the patient. In one embodiment of the method, an electrical stimulation signal is applied to a first neuromuscular structure of the patient. The electrical stimulation signal is communicated through a neural pathway to a second pelvic neuromuscular structure of the patient. The second neuromuscular structure is stimulated in response to the electrical stimulation signal communicated through the neural pathway. The pelvic condition is treated in response to the stimulation of the second pelvic neuromuscular structure.

This Summary is provided to introduce a selection of concepts in a simplified form that are further described below in the Detailed Description. This Summary is not indented to identify key features or essential features of the claimed subject matter, nor is it intended to be used as an aid in determining the scope of the claimed subject matter.

### BRIEF DESCRIPTION OF THE DRAWINGS

FIG. 1 is a schematic, pictorial view of an exemplary implantable electronic stimulator device in accordance with embodiments of the invention.

FIG. 2 is a side plan view of an exemplary electronic stimulator device, in accordance with embodiments of the invention.

FIG. 3 is a flowchart illustrating a method of treating a pelvic condition of a patient in accordance with embodiments of the invention.

FIG. 4 is a schematic diagram of a pelvic region of a patient illustrating steps of the method in accordance with embodiments of the invention.

### DETAILED DESCRIPTION OF ILLUSTRATIVE EMBODIMENTS

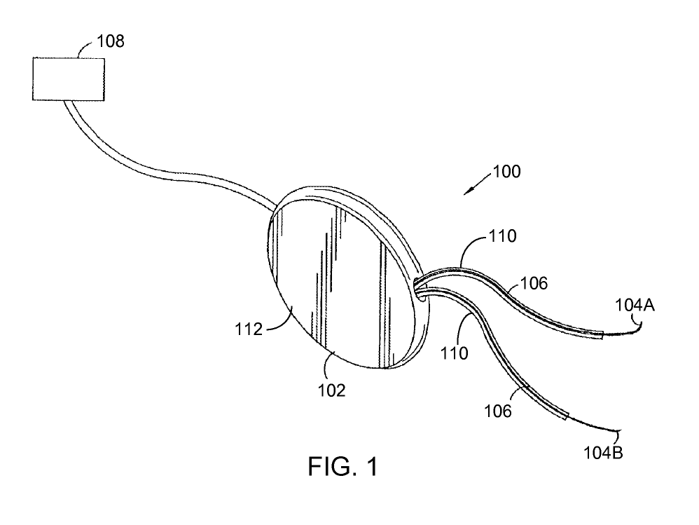

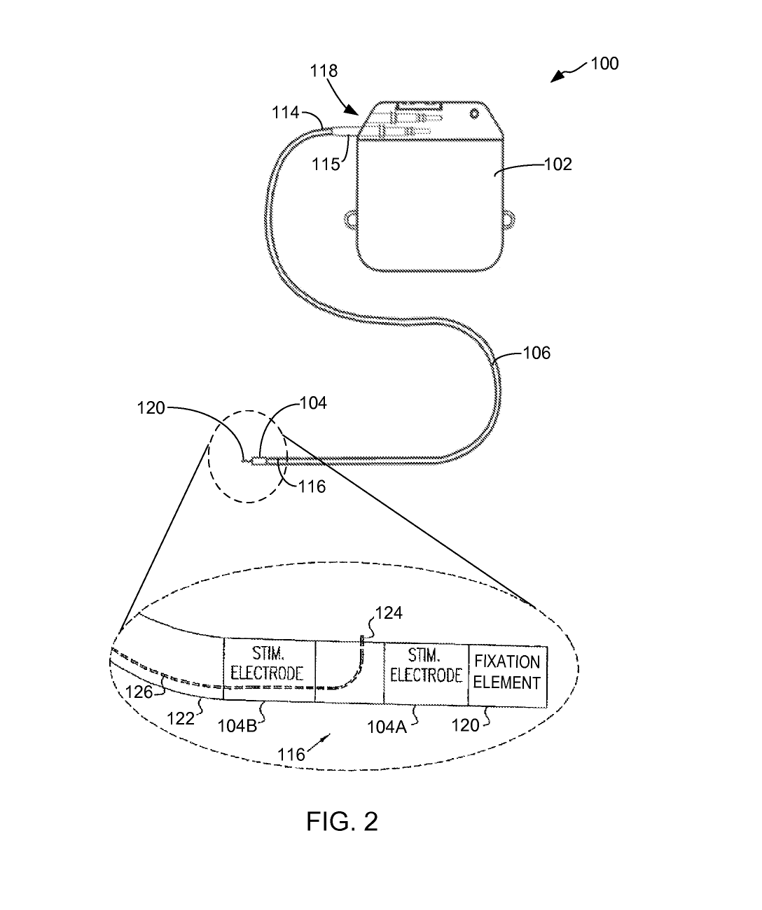

Embodiments of the present invention generally relate to the treatment of a pelvic condition of a patient through the indirect stimulation of a pelvic neuromuscular structure of the patient. Before discussing various embodiments of the method, exemplary implantable electronic stimulator devices will be described with reference to FIGS. 1 and 2.

FIG. 1 is a schematic, pictorial view of an exemplary implantable electronic stimulator device 100, in accordance with embodiments of the invention. Device 100 is configured for implantation into the pelvic region of a patient, as described in detail below, for use in providing muscle and/or nerve stimulation that is used to control and/or treat a pelvic condition, such as pelvic pain, urinary incontinence and other pelvic conditions. In one embodiment, the device 100 comprises a control unit 102 and one or more electrodes, generally referred to as 104, such as electrodes 104A and 104B. Electrodes 104 are coupled to the control unit 102 by leads 106. In one embodiment, the device 100 includes at least one physiological sensor 108, such as a miniature ultrasound transducer, one or more accelerometers, an electromyography recording apparatus, a pressure transducer or other sensors known in the art.

In one embodiment, the control unit 102 comprises circuitry for sensing electrical signals received by the electrodes 104, such as electromyography (EMG) signals, along with circuitry for processing the signals from the sensor 108. In one embodiment, the control unit 104 comprises circuitry for applying electrical stimulation waveforms to one or more of the electrodes 104. The electrical stimulation waveforms are designed to control and/or treat the desired condition of the pelvic region.

In one embodiment, the electrodes 104 are flexible intramuscular-type wire electrodes, approximately 1-35 millimeters long and 50-100 microns in diameter, in order to minimize patient discomfort. In one embodiment, the electrodes 104 comprise a spiral hook, as known in the art, so that they can be easily and permanently anchored in a pelvic muscle of a patient. The wire, from which the electrodes 104 are made, comprises a suitable conductive material, such as a biocompatible metal such as stainless steel, silver, a platinum/iridium alloy (90-10) or a nickel-chromium alloy. The leads 106 have a length that is suitable for the application, such as 5-10 centimeters long, and are surrounded by an insolating jacket 110 typically comprising silicone, polyurethane or and other flexible, biocompatible insolating material. An optional additional wire (not shown) inside the jacket 110 can serve as an antenna for the purpose of wireless communications with the device 100, in accordance with known methods.

In one embodiment, the control unit 102 comprises a circuitry for processing electrical signals received from the electrodes 104 or the sensor 108 and/or for applying an electrical waveform to one or both of the electrodes 104. In one embodiment, the circuitry is contained in a case 112 made of titanium or other suitable biocompatible metal. Typically, the case 112 is about 20 millimeters in diameter and 4 millimeters thick. For some applications, the case 112 serves as a ground electrode for the electrodes 104 when they are sensing or stimulating in a monopolar mode. Alternatively, the case 112 may comprise metal coated with a layer of biocompatible plastic, such as polymethyl methacrylate (PMMA) or silicon.

Although two electrodes 104A and 104B and one sensor 108 are shown attached to the control unit 102 in FIG. 1, it is possible to use only a signal electrode 104 as illustrated in FIG. 2, which is a side plan view of an exemplary electronic stimulator device 100, in accordance with another embodiment of the invention. Except with respect to the difference described below, the embodiment of the device 100 shown in FIG. 2 is generally similar to the embodiments shown in FIG. 1, and techniques described herein with respect to one of these configurations can generally be applied to the other configuration. Accordingly, elements in FIG. 2 that are labeled with the same or similar numbers as that used in FIG. 1, generally correspond to the same or similar elements.

One embodiment of the device 100 shown in FIG. 2 comprises a control unit 102, at least one electrode 104 and a lead 106 connecting electrode 104 to the control unit 102. The lead 106 includes a proximal end 114 that is coupled to the control unit 102 via a connector 115 and a distal end 116 at which the electrode 104 is located. Additional leads 106 or sensors 108 may be coupled to the control unit 102 at a suitable interface, such as interface 118.

The electrode 104 can be anchored to a pelvic floor muscle of the patient by means of a fixation element 120, such as a helix, spiral hook or other anchor known in the art, as shown in the magnified schematic illustration of the distal end 116 of the lead 106 provided in FIG. 2. The helix or spiral hook 120 can be embedded within the external urinary or anal sphincter to anchor the lead 106 thereto. In one embodiment, the fixation element 120 operates to provide electrical contact between the muscle and one or more stimulation electrodes 104A and 104B disposed on a silicone casing 122 of the lead 106.

In one embodiment, the electrodes, generally referred to as 104, are approximately 3 millimeters in length, but can be much longer, such as less than about 80 millimeters in length, for example. The electrodes 104 are typically separated by approximately 3 millimeters along the length of the lead 106. In the same between the electrodes 104A and 104B a tip 124 of an EMG wire 126 may protrude approximately 100 microns through the casing 124, for those applications in which EMG sensing is desirable. Typically, the diameter of the wire 126 is approximately 50 microns, and the diameter of the casing 124 is approximately 1.5 millimeters.

As with the device 100 illustrated in FIG. 1, one embodiment of the device 100 illustrated in FIG. 2 comprises circuitry for applying electrical stimulation waveforms to the muscular tissue, in which the fixation element 120 is embedded, and/or for sensing electrical signals received by the electrodes 104, in accordance with conventional implantable electronic stimulator devices known in the art.

Embodiments of the invention are generally directed to a method of treating a pelvic condition of a patient through the indirect stimulation of a pelvic neuromuscular structure of the patient. The indirect stimulation of a pelvic neuromuscular structure is accomplished by taking advantage of the existence of cross reflex mechanisms or cross-talk between neural pathways of pelvic neuromuscular structures of the patient. As used herein, the term “neuromuscular structure” describes muscle tissue or nerves.

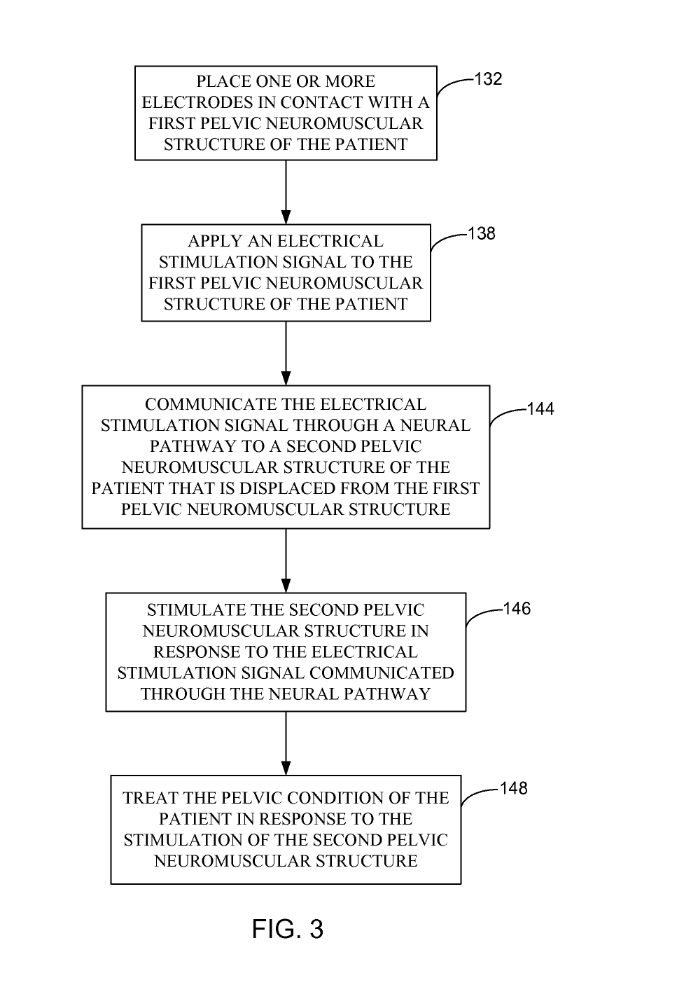

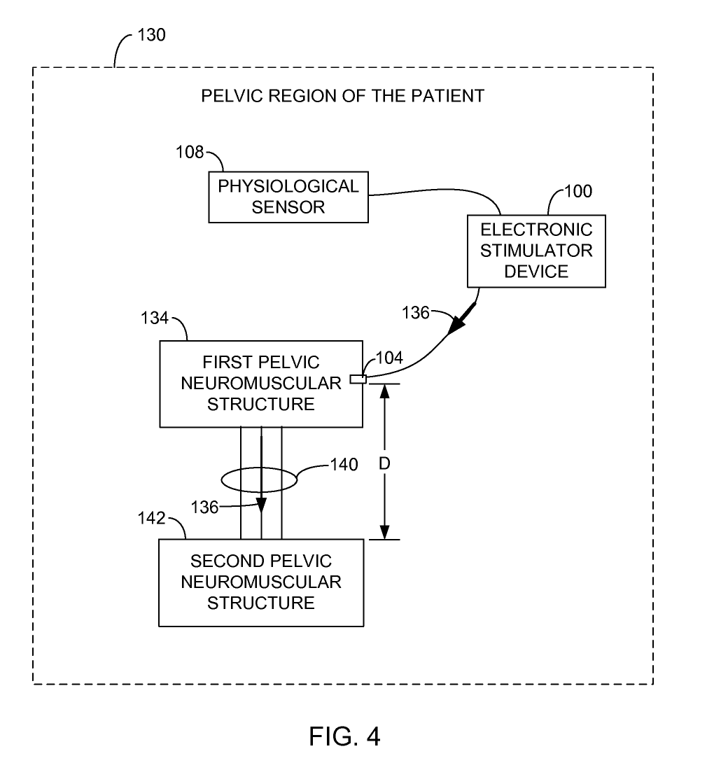

The general method of the present invention, in accordance with various embodiments of the invention, will be described with reference to the flowchart of FIG. 3 and the schematic diagram of a pelvic region 130 of a patient provided in FIG. 4. At step 132, one or more electrodes 104 of the stimulation device 100 are placed in contact with a first pelvic neuromuscular structure 134 of the patient. This includes embedding or implanting the one or more electrodes 104 in the first pelvic neuromuscular structure 134 of the patient. The electrodes 104 are configured to receive a electrical stimulation signal or waveform 136 from a stimulator device in accordance with conventional techniques. In one embodiment, the stimulator device comprises device 100 described above.

At step 138, the electrical stimulation signal or waveform 134, generated by the device 100 or other stimulator device, is applied to the first pelvic neuromuscular structure of the patient through the one or more electrodes 104. The electrical stimulation signal 136 is then communicated through a neural pathway 140 (e.g., afferent and/or efferent neural pathways) from the first pelvic neuromuscular structure 134 to a second pelvic neuromuscular structure 142 of the patient, as indicated at step 144. The location of the one or more electrodes 104 in or on the first pelvic neuromuscular structure 134 is displaced from the second pelvic neuromuscular structure by a distance D. The distance D can cross organs and their borders. In one embodiment, the distance D is greater than 1.0 cm. In accordance with another embodiment, the distance D is greater than approximately a few centimeters, such as 2-3.0 cm. In yet another embodiment, the distance D is 2-10 cm.

At step 146, the second pelvic neuromuscular structure is indirectly stimulated in response to the electrical stimulation signal communicated through the neural pathway 140. The condition or symptom of the patient is then treated, at least in part, in response to the stimulation signal 136 that is indirectly applied to the second pelvic neuromuscular structure 142 through the neural pathway 140, as indicated at step 148.

In accordance with exemplary embodiments, the first pelvic neuromuscular structure 134 of the patient comprises an anal neuromuscular structure and the second pelvic neuromuscular structure 142 of the patient comprises a urinary neuromuscular structure. Embodiments of the anal neuromuscular structure include an external anal sphincter muscle, an internal anal sphincter muscle and a pudendal nerve of the patient. The pudendal nerve, which innervates the external anal sphincter muscle and the external urinary sphincter muscle, forms at least a component of the neural pathway. In one embodiment, the anal neuromuscular structure containing the pudendal nerve is remote from the urinary neuromuscular structure that is being indirectly stimulated, by approximately the distance D.

Exemplary embodiments of the condition or symptom that may be treated by the direct application of the stimulation signal 136 to the first pelvic neuromuscular structure 134 and the indirect application of the stimulation signal 136 to the second pelvic neuromuscular structure 142 include urinary incontinence, urinary urgency and frequency, urinary retention, pelvic pain, endometriosis, fecal incontinence, constipation, sexual dysfunction, interstitial cystitis, chronic prostatitis and other pelvic conditions or symptoms.

Embodiments of the electrical stimulation signal or waveform 136 that is generated by the stimulator device 100 and applied to the first pelvic neuromuscular structure 134 through the one or more electrodes 104 may take on different forms depending on the particular application or pelvic condition being treated. For example, the electrical stimulation signal 136 may be monophasic or biphasic. In one embodiment, the electrical stimulation signal 136 comprises a plurality of electrical pulses. The electrical pulses may have a range of current and voltage amplitudes, duty cycles, frequencies (i.e., pulse repetition rates) and duration of application. It has been found that pulse frequencies in the range of 5 and 200 Hz are effective in engendering contraction in pelvic muscles, such as the levator ani muscle, but it may be desirable to use frequencies outside of this range. to In accordance with preferred embodiments, the electrical stimulation signal comprises a biphasic square wave having the following characteristics:

Current: 2-100 mA, preferably 2-4 mA;

Voltage: 1-15 V, preferably 1-3V;

Pulse width: 10 μs-2 ms, preferably 100-200 μs and variable in increments of 0.1 ms; and

Pulse repetition rate: 5-50 Hz, 50-1000 Hz, 12-25 Hz, preferably 10-30 Hz.

In one embodiment of the method, the application of the electrical stimulation signal 136 (step 138) is terminated after a predetermined period of time. In one embodiment, the predetermined period of time is 5-30 seconds. In another embodiment, the predetermined period of time is less than 6 hours. Other stimulation periods can be used depending on the particular application.

In one embodiment, the application of the electrical stimulation signal 136 is in response to the sensing of a physiological characteristic of the patient that is indicative of a pelvic condition requiring treatment. In one embodiment, the stimulator device 100 analyzes signals from the sensor 108 that are indicative of a physiological characteristic of the patient. The stimulator device 100 responsively applies the electrical stimulation signal 136 to the first pelvic neuromuscular structure 134 when the signal from the sensor 108 indicates the need to treat a pelvic condition, to which the signal relates. In one embodiment, the stimulator device 100 applies the electrical stimulation signal 136 to the first pelvic neuromuscular structure 134 when the signal from the sensor 108 indicates imminent urinary incontinence (e.g., urge and/or stress incontinence) or bladder retention problems.

In one embodiment, the pelvic condition of the patient that is to be treated through the application of the stimulation signal 136 to the anal neuromuscular structure (134) is urinary or bladder retention. Urinary retention occurs when the patient is unable to release urine from the bladder. This may be caused by either an outflow obstruction or poor bladder contractibility. In each case, the outflow resistance exceeds the pressure generated by contraction of the detrusor muscle that, under normal functioning, pushes the urine out of the bladder. In accordance with this embodiment, the urinary neuromuscular structure (142) of the patient comprises the detrusor muscle. The indirect stimulation of the detrusor muscle in response to the stimulation of the anal neuromuscular structure (134) contracts the detrusor muscle and, thus, increases the contractility of the bladder to treat the urinary or bladder retention condition of the patient.

Chart 1 below shows the pressures of the anal and bladder of the patient in response to the application of the electrical stimulation signal 136 (2 mA, 30 pulses/second, 100 μs pulse width duration) to the external anal sphincter (134) over the period of time identified by the thick solid line. The data illustrate the existence of a direct correlation between the stimulation of the external anal sphincter muscle (134) and the increase in contractility of the bladder. The increase in contractility of the bladder is presumably the result of the indirect stimulation of the detrusor muscle (142) by the electrical stimulation signal 136 communicated through the neural pathway 140 (pudendal nerve) from the external anal sphincter muscle 134.

Embodiments of the electrical stimulation signal for the treatment of urinary retention preferably comprise electrical pulses having a current amplitude of 2.0-4.0 mA, stimulation duration of 5-30 seconds, a pulse frequency of 10-30 Hz and a pulse duration of 100-200 μsec. In one embodiment, the electrical stimulation signal has a current amplitude of 2 mA, a stimulation duration of 10 seconds, a pulse frequency of 30 Hz and pulse duration of 100 μsec.

In accordance with another embodiment, the pelvic condition of the patient that is to be treated through the application of the stimulation signal 136 to the anal neuromuscular structure (134) is urinary incontinence. Urinary incontinence is the inability of the patient to control the release of urine from his or her bladder. Embodiments of the invention include the treatment of various types of urinary incontinence including stress incontinence, urge incontinence and mixed incontinence. In each case, the pressure on the bladder exceeds the outflow resistance produced by the internal and/or external urinary sphincter muscles resulting in involuntary urination.

In accordance with this embodiment, the urinary neuromuscular structure (142) of the patient comprises the internal urinary sphincter muscle and/or the external urinary sphincter muscle. The electrical stimulation signal 136 that is applied to the anal neuromuscular structure (134) in step 132 is communicated to the internal and/or external urinary sphincter muscle (142) at least partially through the pelvic, hypogastric and/or pudendal nerve (i.e., neural pathway 140). In response to this indirect application of the electrical stimulation signal 136 to the internal and/or external urinary sphincter muscles (142), the internal and/or external urinary sphincter muscles (142) contract and restrict the flow of urine through the urethra of the patient thereby treating the urinary incontinence condition.

Embodiments of the electrical stimulation signal 136 for the treatment of urinary incontinence preferably comprise electrical pulses having a current amplitude of 3.0-4.0 mA, a voltage amplitude of 1-3 V, a stimulation duration that is intermittent or continuous over a period of several seconds to several hours (e.g., 6 hours), a pulse frequency of 12-25 Hz and a pulse duration of 10-200 μsec. In one embodiment, the electrical stimulation signal has a current amplitude of 3 mA, a voltage amplitude of 3 V, a pulse frequency of 25 Hz and pulse duration of 200 μsec.

One advantage of the method of the present invention is the ability to place the electrode lead(s) 104 of the stimulator device 100 at a location of the patient that is, for example, easier to access (i.e., less intrusive), more comfortable for the patient, easier to monitor, easier to maintain, etc. For example, it is far less intrusive to the patient to implant an electrode in the external anal sphincter muscle to treat, for example, urinary incontinence or bladder retention, than it is to implant an electrode in the internal urinary sphincter muscle or the detrusor muscle of the patient.

In one embodiment, the first pelvic neuromuscular structure 134 of the patient comprises a urinary neuromuscular structure of the patient, embodiments of which are described above, and the second pelvic neuromuscular structure 142 of the patient comprises an anal neuromuscular structure of the patient, embodiments of which are described above. In accordance with this embodiment, stimulation signals are indirectly applied to the anal neuromuscular structure through the stimulation of the urinary pelvic neuromuscular structure using the neural pathway (e.g., pudendal nerve branches). Exemplary conditions or symptoms that may be treated using this method include fecal incontinence, constipation, endometriosis-induced dysmenorrhea, erectile dysfunction, irritable bowel syndrome, vulvodynia. and other pelvic conditions or symptoms.

Although the present invention has been described with reference to preferred embodiments, workers skilled in the art will recognize that changes may be made in form and detail without departing from the spirit and scope of the invention. For example, the embodiments of the electrical stimulation signal described above are exemplary and other electrical stimulation signals may be used in order to provide the desired treatment of a pelvic condition of a patient.

1. A method of treating a pelvic condition of a patient comprising:
applying an electrical stimulation signal to a first pelvic neuromuscular structure of the patient;
communicating the electrical stimulation signal through a neural pathway to a second pelvic neuromuscular structure of the patient;
stimulating the second pelvic neuromuscular structure in response to the electrical stimulation signal communicated through the neural pathway; and
treating the pelvic condition in response to the stimulation of the second pelvic neuromuscular structure.

2. The method of claim 1, wherein the pelvic condition of the patient is selected from the group consisting of urinary incontinence, urinary urgency, urinary retention, pelvic pain, endometriosis, fecal incontinence, constipation, sexual dysfunction, interstitial cystitis and chronic prostatitis.

3. The method of claim 1, wherein the first pelvic neuromuscular structure is displaced a distance of greater than approximately 2-3.0 cm from the second pelvic neuromuscular structure.

4. The method of claim 1, wherein:
the first pelvic neuromuscular structure comprises an anal neuromuscular structure of the patient selected from the group consisting of the internal anal sphincter, the external anal sphincter and the pudendal nerve proximate the anal canal; and
the second pelvic neuromuscular structure comprises a urinary neuromuscular structure of the patient selected from the group consisting of the internal urinary sphincter, the external urinary sphincter and the detrusor muscle.

5. The method of claim 4, wherein the pelvic condition of the patient is selected from the group consisting of urinary incontinence, urinary urgency and urinary retention.

6. The method of claim 1, wherein:
the first pelvic neuromuscular structure comprises a urinary neuromuscular structure of the patient selected from the group consisting of the internal urinary sphincter, the external urinary sphincter and the detrusor muscle; and
the second pelvic neuromuscular structure comprises an anal neuromuscular structure of the patient selected from the group consisting of the internal anal sphincter, the external anal sphincter and the pudendal nerve proximate an anal canal.

7. The method of claim 6, wherein the pelvic condition of the patient is selected from the group consisting of fecal incontinence and constipation.

8. The method according to claim 1, further comprising providing for the electrical stimulation signal a plurality of electrical pulses.

9. The method according to claim 8, wherein the plurality of electrical pulses each have a pulse width selected from the group consisting of 10 μs-2 ms and 100-200 μs.

10. The method according to claim 8, wherein the electrical pulses have a pulse repetition rate selected from the group consisting of 5-50 Hz, 50-1000 Hz, 10-30 Hz and 12-25 Hz.

11. The method according to claims 8, wherein the plurality of electrical pulses each have a current selected from the group consisting of 2-100 mA and 2.0-4.0 mA.

12. The method according to claim 1, further comprising implanting an electrode in contact with the first pelvic neuromuscular structure, wherein the electrical stimulation signal is applied to the first pelvic neuromuscular structure of the patient through the electrode.

13. The method of claim 12, further comprising:
implanting a stimulator device in the patient; and
generating the electrical stimulation signal using the stimulator device.

14. The method of claim 1, further comprising:
sensing a physiological characteristic of the patient indicative of a condition selected from the group consisting of imminent urge incontinence, imminent stress incontinence and bladder retention; and
applying the electrical stimulation signal in response to the sensing of the physiological characteristic of the patient.

15. A method of treating a pelvic condition of a patient comprising:
applying an electrical stimulation signal to an anal neuromuscular structure of the patient; and
contracting a urinary muscle of the patient in response to the application of the electrical waveform to the anal neuromuscular structure of the patient.

16. The method according to claim 15, wherein:
the anal neuromuscular structure is selected from the group consisting of an internal anal sphincter, an external anal sphincter and a pudendal nerve proximate an anal canal of the patient; and
the urinary muscle of the patient is selected from the group consisting of an internal urinary sphincter, an external urinary sphincter and a detrusor muscle of the patient.

17. The method of claim 16, wherein the pelvic condition of the patient is selected from the group consisting of urinary incontinence, urinary urgency and urinary retention.

18. The method according to claim 17, further comprising providing for the electrical stimulation signal a plurality of electrical pulses.

19. A method of treating urinary retention of a patient comprising:
applying an electrical stimulation signal to an anal neuromuscular structure of the patient; and
increasing contractility of a bladder of the patient in response to the application of the electrical waveform to the anal neuromuscular structure.

20. The method of claim 19, wherein increasing the contractility of the bladder of the patient comprises contracting a detrusor muscle of the patient in response to the application of the electrical waveform to the anal neuromuscular structure.
